(19) United States
(12) Patent Application Publication
(10) Pub. No.: US 2025/0345506 A1
(43) Pub. Date: Nov. 13, 2025

(54) DRAINAGE BOTTLE MONITORING SYSTEM AND METHOD THEREOF

(71) Applicant: CoProg INC., Taipei (TW)

(72) Inventor: Kuan-Yin Yu, Taipei (TW)

(21) Appl. No.: 18/781,846

(22) Filed: Jul. 23, 2024

(30) Foreign Application Priority Data
May 13, 2024 (TW) 113117523

(51) Int. Cl. A61M 1/00

(52) U.S. Cl. CPC A61M 1/69; A61M 2202/0014; A61M 2202/04; A61M 2205/18; A61M 2205/3306; A61M 2205/332; A61M 2205/3327; A61M 2205/3382; A61M 2205/3389; A61M 2205/3393; A61M 2205/3561; A61M 2205/502; A61M 2205/581; A61M 2205/584

(57) ABSTRACT

This invention provides a drainage bottle monitoring system and method thereof. The method includes a measurement device and a monitoring device. The measurement device includes an optical sensor, a weight sensor, and a transmission device. The monitoring device includes a computing processor and a warning notifier. The optical sensor has a receiver-transmitter unit to detect the liquid level of the drainage bottle and obtain liquid level information. The weight sensor measures the weight of the drainage bottle and the weight of the liquid in the bottle to obtain weight information. The transmission device is used to transmit liquid level and weight information. The computing processor calculates the actual liquid volume of the drainage bottle and liquid error. The warning notifier issues a alert message when the liquid level or actual liquid volume exceeds a preset value.

### CROSS-REFERENCE TO RELATED APPLICATION

This application claims the priority benefit of Taiwan application no. 113117523, filed on May 13, 2024, the full disclosure of which is incorporated herein by reference.

### BACKGROUND

### Technical Field

This disclosure relates to a drainage bottle monitoring system and method thereof, specifically related to a drainage bottle monitoring system and method that uses optical means supplemented by weight sensing to monitor the volume within the drainage bottle.

### Description of Related Art

Thoracic drainage is a common treatment method in the medical field, typically used to manage the accumulation of gas or liquid within the thoracic cavity. This treatment is particularly common in cases of pneumothorax, empyema, or hemothorax, especially after thoracic surgery. In these situations, the patient's pleural cavity may accumulate excessive air or liquid, leading to lung compression and symptoms such as difficulty breathing. The primary purpose of thoracic drainage is to remove these accumulations of liquid and gas, restore the normal negative pressure state of the thoracic cavity, thereby aiding lung expansion, reducing breathing difficulties, and also monitoring for conditions such as bleeding or alveolar air leaks.

However, in clinical practice, current drainage systems have some issues. For example, the method of securing the chest tube is primarily using adhesive tape, and the thoracic drainage bottle is usually placed on a general iron drainage stand or on the floor beside the patient's bed. This placement method poses risks such as tipping over, pulling, or chest air leakage, all of which can affect the treatment's effectiveness and potentially harm the patient.

Additionally, the size of the drainage bottles used by different hospitals varies, resulting in a lack of universality in existing drainage systems across different hospitals. This may require customization or adjustments for each hospital's specific bottle size, and different sizes of bottles may need different drainage stands or securing methods, increasing the difficulty of setup and operation.

Furthermore, medical staffs usually need to closely monitor the operation of the thoracic drainage system, observing the increase in liquid and checking for signs of chest air leaks. However, the current practice often relies on medical staff visually observing the changes in the liquid in the drainage bottle and manually recording the relevant data. This method requires staff to regularly check the drainage bottle and record each liquid change, which is prone to human error and time-consuming. Due to the need for manual recording, medical staff might miss important records due to fatigue or distraction, or make errors or omissions in recording, affecting the accurate assessment of the patient's condition. Additionally, relying on visual observation has subjectivity and limitations. Medical staff's observations may be influenced by personal subjective consciousness, vision differences, or the work environment, potentially leading to inaccurate or inconsistent assessments of liquid increase or chest air leaks.

Furthermore, bubbles can cause reading errors, further affecting the assessment of the patient's condition.

Accordingly, developing a drainage bottle monitoring system and method that can solve the aforementioned problems has become an issue to be addressed in the relevant technical field.

### SUMMARY

In one aspect, the present invention is directed to a drainage bottle monitoring system and method thereof. The drainage bottle monitoring system is provided. The drainage bottle monitoring system for monitoring the accumulated liquid in a drainage bottle. The drainage bottle monitoring system comprises a measuring device and a monitoring device. The measuring device has a housing portion for accommodating the drainage bottle. The measuring device comprises an optical sensor, a weight sensor, and a transmission device. The monitoring device is communication-connected to the transmission device for receiving the liquid level height information and the weight information. The monitoring device comprises a computing processor and an alert notifier.

The optical sensor is set on sidewalls within the housing portion of the measuring device. The optical sensor has a transmitting-receiving unit for detecting the liquid level height of the liquid in the drainage bottle, thereby obtaining liquid level height information. Each transmitting-receiving unit comprises a light emitting unit and multiple light receiving units. The weight sensor is set at the bottom of the housing portion of the measuring device for measuring a weight of the drainage bottle and the weight of the liquid in the drainage bottle, thereby obtaining weight information. The transmission device is set within the measuring device and signal-connected to the optical sensor and the weight sensor for transmitting the liquid level height information and the weight information. The computing processor calculates the actual liquid volume in the drainage bottle and a liquid error based on the liquid level height information and the weight information. The alert notifier is communication-connected to the computing processor and issues an alert message when the liquid level height or the actual liquid volume exceeds a preset value.

According to another embodiment, the alert notifier comprises an indicator light and a buzzer, and the indicator light emits a red light and the buzzer emits a warning sound when the liquid level height or the actual liquid volume exceeds a preset value.

According to another embodiment, the weight sensor is a load cell.

According to another embodiment, the actual liquid volume is calculated by subtracting the weight of the drainage bottle from the weight of the liquid in the drainage bottle, and then dividing by a density of the liquid.

According to another embodiment, the liquid error is obtained by subtracting the actual liquid volume from the liquid level height of the drainage bottle.

According to another embodiment, further comprises a display device, which is communication-connected to the computing processor for displaying the total liquid capacity of the drainage bottle, the accumulated liquid capacity from the previous hour, time, error, and battery power.

According to another embodiment, the transmitting-receiving unit is formed by a plurality of vertically stacked phototransistors.

According to an embodiment, a drainage bottle monitoring method, comprises the following steps. First, housing a drainage bottle in a housing portion of a measuring device. Second, detecting the liquid level height of the drainage bottle with an optical sensor of the measuring device to obtain liquid level height information. Finally, issuing an alert message from an alert notifier of a monitoring device when the liquid level height exceeds a preset value.

According to another embodiment, further comprise the following steps. First, measuring the weight of the liquid in the drainage bottle with a weight sensor of the measuring device to obtain weight information. Second, calculating the actual liquid volume and a liquid error of the drainage bottle based on the liquid level height information and the weight information with a computing processor of the measuring device. Finally, issuing an alert message from the alert notifier of the monitoring device when the actual liquid volume exceeds a preset value.

According to another embodiment, the actual liquid volume is calculated by subtracting the weight of the drainage bottle from the weight of the liquid in the drainage bottle, and then dividing by the density of the liquid.

In summary, the effectiveness of the present invention lies in providing a drainage bottle monitoring system capable of real-time monitoring of the liquid level height and liquid weight in the drainage bottle, and automatically calculating the actual volume and error of the liquid. When the liquid amount exceeds a preset value, the alert notifier will issue an immediate warning to remind the operator to take action. Additionally, the system can display information such as the total liquid capacity of the drainage bottle, accumulated liquid amount, time, and battery power, allowing medical staff to manage the patient's drainage situation more conveniently, thereby improving the efficiency and quality of medical work.

### BRIEF DESCRIPTION OF THE DRAWINGS

In order to make the above-described technology of this disclosure, as well as other aspects, features, advantages, and embodiments, more apparent and understandable, the description of the accompanying drawings is provided as follows.

FIG. 1 is a system block diagram of a drainage bottle monitoring system according to one embodiment of the present invention.

FIG. 2 is a top view of a measuring device of a drainage bottle monitoring system according to one embodiment of the present invention.

FIG. 3 is a cross-sectional view of the measuring device of the drainage bottle monitoring system in FIG. 2.

FIG. 4 is a cross-sectional view of the weight sensor of the drainage bottle monitoring system in FIG. 3.

FIG. 5 is a schematic diagram of the drainage bottle monitoring system in FIG. 3 measuring the liquid level of a drainage bottle.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

To provide a more detailed explanation of various embodiments of the present invention, the following is accompanied by drawings for illustration. It should be understood that when an element is referred to as being “connected” or “disposed” on another element, it can indicate that the element is directly located on the other element, or there may be an intermediate element connecting the element to the other element. Conversely, when an element is described as being “directly on” or “directly connected to” another element, it is explicitly defined that there is no intermediate element involved.

The terms “comprising,” “including,” or any variations thereof are intended to encompass non-exclusive inclusion, so that processes, methods, products, or devices comprising a series of elements not only include those elements explicitly listed, but also include other elements inherently present in such processes, methods, products, or devices. In the absence of further limitations, elements specified by the phrase “comprising a . . . ” do not exclude the presence of additional identical elements in processes, methods, products, or devices that include the specified elements.

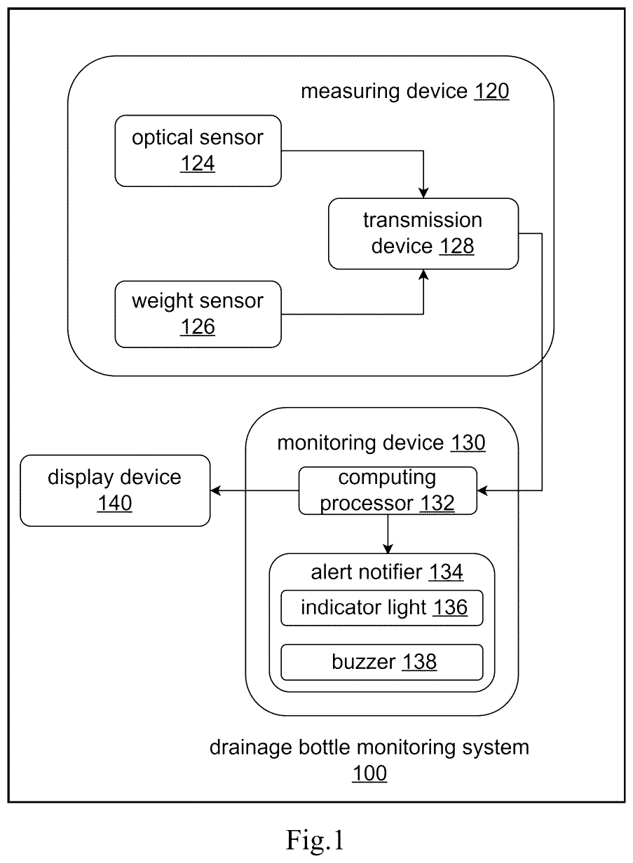

FIG. 1 is a system block diagram of a drainage bottle monitoring system according to one embodiment of the present invention. In FIG. 1, a drainage bottle monitoring system 100 for monitoring the accumulated liquid in a drainage bottle 110 (shown in FIG. 4) is provided. The drainage bottle monitoring system 100 comprises a measuring device 120 and a monitoring device 130. The drainage bottle 110 is typically made of a transparent material. The drainage bottle 110 comprises structures such as a guide tube, a guide port, and an exhaust port for collecting liquid from the patient's body.

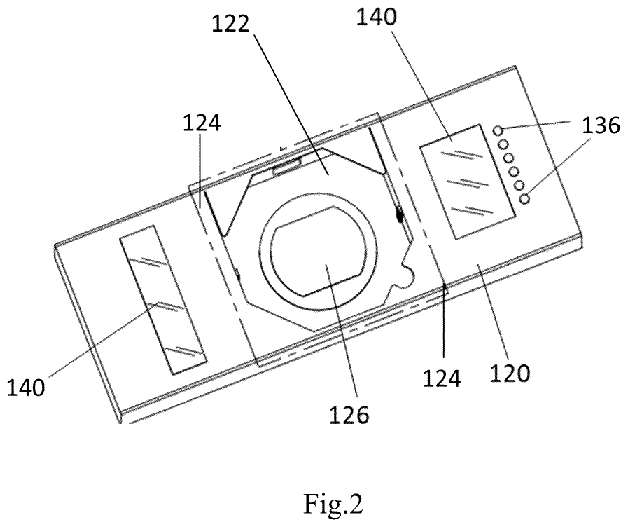

In FIG. 1, the measuring device 120 comprises an optical sensor 124, a weight sensor 126, and a transmission device 128. FIG. 2 is a top view of a measuring device of a drainage bottle monitoring system according to one embodiment of the present invention. In FIG. 2, the measuring device 120 has a housing portion 122 for accommodating the drainage bottle 110, the optical sensor 124 and the weight sensor 126. The housing portion 122 is a round hole that can safely accommodate the drainage bottle 110 disposed on the weight sensor 126 and provide appropriate support to the drainage bottle 110.

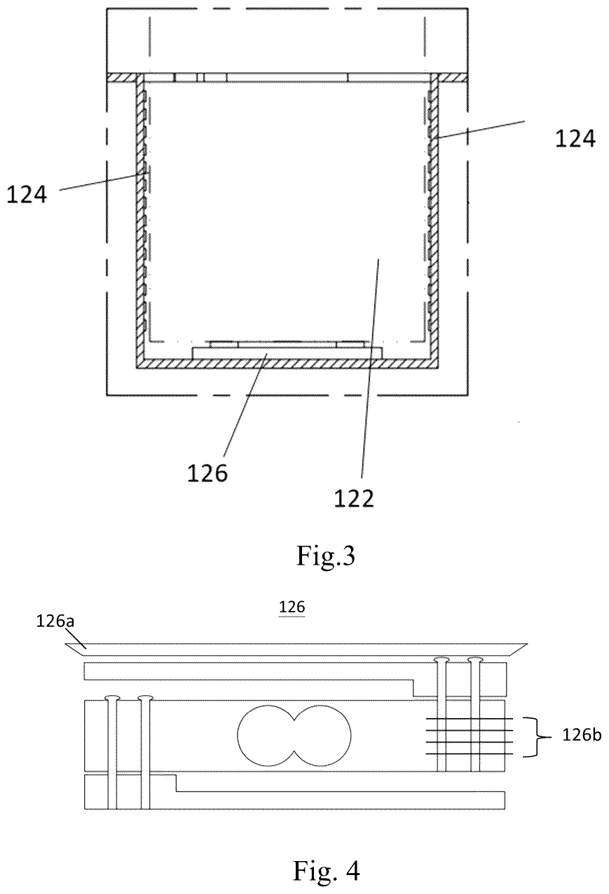

FIG. 3 is a cross-sectional view of the measuring device of the drainage bottle monitoring system in FIG. 2. In FIG. 2 and FIG. 3, the weight sensor 126 is disposed at the bottom of the housing portion 122 of the measuring device 120, with the drainage bottle 110 placed on top of the weight sensor 126. The primary function of the weight sensor 126 is to measure the weight of the drainage bottle 110 and the weight of the liquid within the drainage bottle 110 to provide relevant weight information. When the drainage bottle 110 (not shown in FIG. 3) is placed on the weight sensor 126, the weight sensor 126 senses the weight of the drainage bottle 110 and converts this weight into an electrical signal output.

FIG. 4 is a cross-sectional view of a weight sensor of a drainage bottle monitoring system in FIG. 3. In FIG. 4, the weight sensor 126 is illustrated as a load cell, for example. A load cell is a sensor used to measure the force exerted on an object, capable of directly measuring force or weight thereon. The sensor of the load cell measures the deformation caused by the force or weight applied in the load cell, thereby converting mechanical force into an electronic signal. This device uses a combination of strain gauges and a bridge circuit to generate a voltage output proportional to the applied force on the load cell. When the drainage bottle 110 is placed on the tray 126a of the load cell, the strain gauge in the load cell is deformed under the pressure. This deformation causes a change in resistance, which is converted into an electrical signal and transmitted through the sensor cable 126b to the transmission device 128.

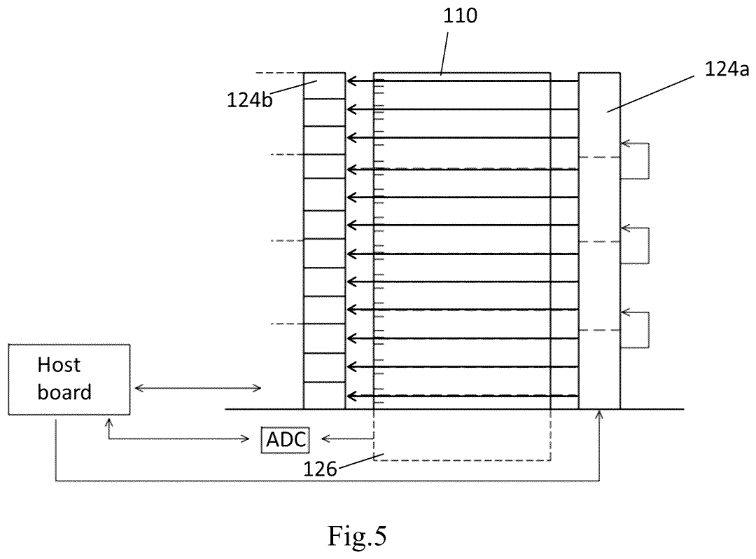

FIG. 5 is a schematic diagram of a drainage bottle monitoring system measuring the liquid level of a drainage bottle according to one embodiment of the present invention. In FIG. 3 and FIG. 5, the optical sensor 124 is disposed on sidewalls of the housing portion 122 of the measuring device 120 to effectively monitor the liquid level height in the drainage bottle 110. The optical sensor 124 has multiple transmitting-receiving units. The multiple transmitting-receiving units are vertically stacked to ensure a broader measurement range for the liquid level height in the drainage bottle 110. This design enables more sensitive detection of changes in the liquid level height and provides more accurate measurement results.

In FIG. 5, each of the multiple transmitting-receiving units comprises a light-emitting unit 124a and several vertically-stacked light-receiving units 124b. The light-emitting unit 124a and the light-receiving units 124b are positioned on the sidewalls of the housing portion 122. The light-emitting unit 124a, such as an LED or a laser diode, emits light that passes through the drainage bottle 110. The light-receiving units 124b, which can be photodiodes or photoresistors, detect the light that has passed through the drainage bottle 110. When the optical sensor 124 operates, the light-emitting unit 124a sends light through the liquid inside the drainage bottle 110. The light-receiving units 124b then receive this light. Due to the liquid's presence, the light intensity is reduced. The light-receiving units 124b determine the liquid level height based on the received light's intensity, allowing the optical sensor 124 to gather information about the liquid level.

To measure the liquid level more accurately, multiple light-emitting units 124a are arranged vertically to ensure the emitted light beams cover the entire drainage bottle 110. Correspondingly, multiple light-receiving units 124b are vertically arranged to capture all light signals, ensuring precise liquid level measurement. This vertical arrangement of light-emitting and light-receiving units provides a broader measurement range and enables the sensor to detect minor changes in the liquid level with high precision. By stacking the light-receiving units 124b vertically, the sensor can monitor a larger vertical range within the drainage bottle with a more precise measurement, ensuring continuous tracking of the liquid level as it changes. This setup offers real-time, accurate data, enhancing the overall performance of the drainage bottle monitoring system.

In FIG. 1, The transmission device 128 is disposed within the measuring device 120 and is signal-connected to the optical sensor 124 and the weight sensor 126 for transmitting the liquid level height information and weight information to the monitoring device 130.

Continuing with reference to FIGS. 1 and 2, the monitoring device 130 is communication-connected to the transmission device 128 to receive the liquid level height information and weight information. The monitoring device 130 comprises a computing processor 132 and an alert notifier 134.

The function of the computing processor 132 is to perform corresponding data processing and calculations based on the collected liquid level height information and weight information to derive the actual liquid volume and liquid error in the drainage bottle 110. Specifically, the computing processor 132 first uses the collected weight information to calculate the actual liquid volume. The actual liquid volume is obtained by subtracting the weight of the drainage bottle 110 from the weight of the liquid in the drainage bottle 110, and then dividing by a density of the liquid.

Simultaneously, the computing processor 132 processes the collected liquid level height information, comparing the liquid level height with the actual liquid volume to calculate the liquid volume error. The liquid error is determined by subtracting the actual liquid volume from the liquid level height, and this error calculation helps the user understand the accurate condition of the liquid in the drainage bottle 110 and make necessary adjustments and management.

The alert notifier 134 is communication-connected to the computing processor 132 and promptly receives the relevant information about the liquid level height and the actual liquid volume. When the alert conditions are triggered, i.e., the liquid level height or the liquid volume exceeds the preset value, the alert notifier 134 activates immediately to notify the user in a timely manner.

The alert notifier 134 comprises an indicator light 136 and a buzzer 138. When the alert conditions occur, the indicator light 136 emits a red light to visually remind the user, while the buzzer 138 emits a sound alarm to audibly draw the user's attention. This dual warning method effectively alerts the user to the presence of a problem, prompting them to take necessary actions promptly.

According to another embodiment, the drainage bottle monitoring system 100 further comprises a display device 140, which is communication-connected to the computing processor. The display device 140 displays the total liquid capacity of the drainage bottle 110, accumulated liquid volume in the previous hour, time, error, or battery power level. The battery power level is displayed only when the drainage bottle monitoring system 100 uses a backup lithium battery.

According to one embodiment, the present invention provides a method for monitoring a drainage bottle, comprising the following steps:
1. The drainage bottle 110 is placed in the housing portion 122 of the measuring device 120.
2. The optical sensor 124 is used to measure the liquid level of the drainage bottle 110 and obtain the liquid level information.
3. When the liquid level exceeds a preset value, an alert message is emitted by the alert notifier 134 to alert the user.

According to another embodiment, the method for monitoring drainage bottle 110 further comprises additional steps involving weight sensing, in addition to the optical sensing mentioned earlier, to provide comprehensive monitoring. The steps are as follows:
1. The weight of the liquid in the drainage bottle 110 is measured by the weight sensor 126 to obtain weight information.
2. The actual liquid volume and liquid error in the drainage bottle 110 are calculated by the computing processor 132 based on the liquid level and weight information.
3. When the actual liquid volume exceeds a preset value, another alert message is emitted by the alert notifier 134 to notify the user.

In summary, the present invention effectively monitors the liquid level and weight of drainage bottles, calculates the actual liquid volume based on this information, thereby providing accurate liquid monitoring and alarm functions. The combination of optical and weight sensors allows for a comprehensive understanding of the liquid status inside the drainage bottle, enabling timely alerts and notifications to medical personnel.

The present invention is disclosed herein with reference to preferred embodiments, but it will be understood by those skilled in the art that the above embodiments are provided for descriptive purposes and not intended to limit the scope of the patent claims of the present invention. Any changes or substitutions equivalent to the above embodiments should be interpreted as being within the spirit or scope of the present invention. Therefore, the scope of protection of the present invention should be determined by the claims set forth above.

What is claimed is:

1. A drainage bottle monitoring system for monitoring the accumulated liquid in a drainage bottle, the system comprising:
a measuring device having a housing portion for accommodating the drainage bottle, and the measuring device comprising:
an optical sensor disposed on sidewalls within the housing portion of the measuring device, having multiple transmitting-receiving units for detecting the liquid level height of a liquid in the drainage bottle, thereby obtaining liquid level height information, wherein each of the transmitting-receiving units comprises a light emitting unit and multiple light receiving units;
a weight sensor disposed at the bottom of the housing portion of the measuring device for measuring a weight of the drainage bottle and a weight of the liquid in the drainage bottle, thereby obtaining weight information; and
a transmission device set within the measuring device and signal-connected to the optical sensor and the weight sensor for transmitting the liquid level height information and the weight information;
a monitoring device communication-connected to the transmission device for receiving the liquid level height information and the weight information, and the monitoring device comprising:
a computing processor for calculating the actual liquid volume in the drainage bottle and a liquid error based on the liquid level height information and the weight information; and
an alert notifier communication-connected to the computing processor and issuing an alert message when the liquid level height or the actual liquid volume exceeds a preset value.

2. The drainage bottle monitoring system of claim 1, wherein the alert notifier comprises an indicator light and a buzzer, and the indicator light emits a red light and the buzzer emits a warning sound when the liquid level height or the actual liquid volume exceeds a preset value.

3. The drainage bottle monitoring system of claim 1, wherein the weight sensor is a load cell.

4. The drainage bottle monitoring system of claim 1, wherein the actual liquid volume is calculated by subtracting the weight of the drainage bottle from the weight of the liquid in the drainage bottle, and then dividing by a density of the liquid.

5. The drainage bottle monitoring system of claim 1, wherein the liquid error is obtained by subtracting the actual liquid volume from the liquid level height of the drainage bottle.

6. The drainage bottle monitoring system of claim 1, further comprising a display device communication-connected to the computing processor for displaying the total liquid capacity of the drainage bottle, the accumulated liquid capacity from the previous hour, time, error, and battery power.

7. The drainage bottle monitoring system of claim 1, wherein the transmitting-receiving unit is formed by a plurality of vertically stacked phototransistors.

8. A drainage bottle monitoring method, comprising the following steps:
housing a drainage bottle in a housing portion of a measuring device;
detecting the liquid level height of the drainage bottle with an optical sensor of the measuring device to obtain liquid level height information;
issuing an alert message from an alert notifier of a monitoring device when the liquid level height exceeds a preset value.

9. The drainage bottle monitoring method of claim 8, further comprising:
measuring the weight of the liquid in the drainage bottle with a weight sensor of the measuring device to obtain weight information;
calculating the actual liquid volume and a liquid error of the drainage bottle based on the liquid level height information and the weight information with a computing processor of the measuring device; and
issuing an alert message from the alert notifier of the monitoring device when the actual liquid volume exceeds a preset value.

10. The drainage bottle monitoring method of claim 9, wherein the actual liquid volume is calculated by subtracting the weight of the drainage bottle from the weight of the liquid in the drainage bottle, and then dividing by the density of the liquid.
